(19) United States
(12) Patent Application Publication
(10) Pub. No.: US 2012/0123012 A1
(43) Pub. Date: May 17, 2012

(54) MICROENCAPSULATED PHOTOINITIATORS AND THE USE THEREOF FOR DENTAL MATERIALS.

(75) Inventors: Volker Rheinberger, Vaduz (LI); Ulrich Salz, Lindau (DE); Jörg Zimmermann, Zurich (CH); Katharina Landfester, Ulm (DE); Ulrich Ziener, Ulm (DE); Marissa Volz, Ulm (DE)

(73) Assignee: Ivoclar Vivadent AG

(21) Appl. No.: 12/312,015

(22) PCT Filed: Oct. 23, 2007
(86) PCT No.: PCT/EP2007/061348

§ 371 Date: Apr. 2, 2010

(30) Foreign Application Priority Data
Oct. 25, 2006 (DE) 102006050153.5

(51) Int. Cl. A61K 6/00; B01J 13/02

(52) U.S. Cl. 522/64; 264/4.7

(57) ABSTRACT

The present invention relates to microcapsules composed of a shell of polymers and of a core comprising photoinitiators which exhibit acylphosphine oxides, their derivatives or amine coinitiators or mixtures of these compounds, and also to the preparation of these microcapsules and to their use for dental materials.

The present invention relates to the preparation of microencapsulated photoinitiators and to their use in dental materials, in particular for use in adhesives, coating materials or cements, and also to a process for the preparation of self-etching, self-conditioning dental materials.

### STATE OF THE ART

Radically polymerizable dental materials, such as, e.g., sealants, dentin/enamel adhesives, fixing materials or filling composites, are generally cured by exposing to light. The reason for this is the simple handling of light-curing materials. They are generally one-component materials, i.e. they do not have to be mixed before use, they have a long processing time and then, if desired, quickly cure on irradiating. Furthermore, they also exhibit a good storage stability at ambient temperature. Because of the tissue compatibility and the satisfactory through curing with pigmented systems, irradiation is today carried out virtually exclusively with light in the wavelength region from 400 to 500 nm. One of the first photoinitiator systems used in radically polymerizable dental materials was the combination of an alpha-diketone and an amine coinitiator (GB 1 408 265). Corresponding dental compositions in which this photoinitiator system was used are claimed, e.g., in U.S. Pat. No. 4,457,818 or U.S. Pat. No. 4,525,256, camphorquinone preferably being used as-diketone. In addition, diketone combinations, such as, e.g., the combination of 1-aryl-2-alkyl-1,2-ethanediones with cyclic diketones, have been described (U.S. Pat. No. 6,204,302).

Photoinitiator systems consisting only of one initiator molecule, “-cleavable initiators”, such as titanocenes, acylphosphonates, acylphosphine oxides or bisacylphosphine oxides, are likewise used in light-curing dental materials. However, titanocenes on their own are not particularly reactive and are accordingly preferably used in combination with amines and/or peroxides (EP 0 334 338). Acylphosphonates, such as, e.g., di(2,6-dimethylphenyl) benzoylphosphonate, are likewise, because of their low through-curing depth or reactivity, preferably used in combination with a second initiator system, such as, e.g., the camphorquinone/amine system (EP 0 336 417). Acylphosphine oxides, such as, e.g., the (2,4,6-trimethylbenzoyl)diphenylphosphine oxide described in DE 2909992, are likewise preferably used in dental composites (EP 0 173 567). A dental composite which can be light-cured using acylphosphine oxide is likewise described in EP 1 236 459, with the difference from EP 0 173 567 that a special mixture of filler particles is used. EP 0 948 955 describes a dental composition in which an initiator combination of acylphosphine oxide, organic peroxide, tertiary amine and aromatic sulfinic acid or a salt thereof is used. An antibacterial adhesive composition is claimed in US 2002/0035169 in which a mixture of acylphosphine oxide and an alpha-diketone is preferably used as initiator system.

Bisacylphosphine oxides and their use as initiators for the photopolymerization of compounds with carbon-carbon double bonds were described for the first time in DE 3443221.

DE 3801511 describes photopolymerizable dental materials which can be cured in two stages. The materials described therein comprise, inter alia, a photoinitiator component I with an absorption maximum of <450 nm and a photoinitiator component II with an absorption maximum of >450 nm, a bisacylphosphine oxide being used as photoinitiator component I and an -diketone being used as photoinitiator component II. A photopolymerizable dental material comprising a bisacylphosphine oxide as photoinitiator is likewise claimed in DE 3837569, a thiol-ene polymerization being initiated with it. Furthermore, bisacylphosphine oxides have also been described in U.S. Pat. No. 5,399,770 (alkylbisacylphosphine oxides), DE 19532358 (alkoxyphenyl-substituted bisacylphosphine oxides) or WO 03/019295 (bathochromic mono- and bisacylphosphine oxides).

A disadvantage of photoinitiator systems formed from an alpha-diketone and an amine is that they are only to a limited extent suitable for use in self-etching, self-conditioning dental restoration materials. Self-etching, self-conditioning dental materials are characterized in that no preconditioning of the dental hard substance is necessary. They include self-etching dentin/enamel adhesives, self-etching coating materials, methacrylate-strengthened glass ionomer cements and self-adhering composites, or also “compomers”. They are generally formed in such a way that they comprise one or more adhesion monomers with an acid functional group, one or more nonacidic comonomers and a photoinitiator system, and also additional additives, such as fillers, or, if appropriate, also solvents. If alpha-diketone/amine photoinitiator systems as described above are used in these systems, the problem arises that amines are protonated and decomposed under acidic conditions and the initiator system thereby loses some of its effectiveness.

Photoinitiators, such as acyl- or bisacylphosphine oxides, which form polymerization-triggering radicals by monomolecular bond cleavage, “Norrish type I cleavage”, exhibit the disadvantage that the carbon-phosphorus bond present in both molecules is easily cleaved by nucleophilic compounds, such as, e.g., water or alcohols (Crivello J. V. and Dietliker K.: “Photoinitiators for free radical cationic & anionic photo-polymerization”, in: Surface Coatings Technology, Bradley G (ed.), 2nd Edition, John Wiley & Sons, 1998). Because of this, the photoinitiator is gradually decomposed, the initiation of the appropriate restoration material is even insufficient and the curing of the material is incomplete. This means that dental materials formed in this way likewise lose their mechanical properties on storing and the corresponding adhesive, the coating material or the fixing cement loses its clinical suitability with time. The microencapsulating of initiators for use in dental materials has already been described, e.g. in U.S. Pat. No. 5,154,762 and WO 03/057792. However, no photoinitiators were microencapsulated in this connection but only redox initiator constituents of chemically curing dental materials consisting of several components with the aim of being able to bring these into one component without these immediately reacting with one another. For use, the capsule has to be mechanically destroyed in order to activate the redox initiator system.

JP 2004330704 A indeed mentions microcapsules with acylphosphine oxides. However, the field of dental technology is not concerned.

DE 19906834 A1 relates to dental glass ionomer cement materials of paste type. These comprise a first paste comprising an α, β-unsaturated carboxylic acid polymer, water and a filler which does not react with the α, β-unsaturated carboxylic acid polymer and a second paste comprising a fluoroaluminosilicate glass powder and a polymerizable monomer not comprising acid groups. One of the two pastes comprises a polymerization catalyst. In order to achieve curing of the material, both pastes are mixed with one another. However, a miniemulsion is not mentioned.

Dental compositions are known from DE 60116142 T2. A two-component system of parts A and B is concerned. Accordingly, the initiators occur alternately in A or B. Radical initiators, e.g. acylphosphine oxides, are used here. Furthermore, the use of peroxides and amines is mentioned. These can be microencapsulated.

One-component systems do not emerge from this citation.

DE 102004020726A1 relates to a process for the preparation of an aqueous dispersion of polymer-encapsulated pigments. This therefore does not involve the encapsulation of initiators.

### OBJECT OF THE INVENTION

It is the object of the invention to make available photoinitiator systems which bring about polymerization in the visible region and which show an improved storage stability in the presence of water, alcohols and acids and which are accordingly especially suitable for self-etching, self-conditioning dental materials, especially for the preparation of self-etching dentin/enamel adhesives, self-etching coating materials and methacrylate-strengthened glass ionomer cements for dental purposes, activation of the initiator system by mechanical destruction of the capsules not being necessary.

Achievement of the Object of the Invention

The object is achieved through microcapsules composed of a shell of polymers and of a core comprising photoinitiators which exhibit acylphosphine oxides, bisacylphosphine oxides, derivatives thereof or amine coinitiators or mixtures of these compounds. The problem of the decomposition of the initiators is accordingly solved according to the invention in such a way that the photoinitiators are protected by microencapsulation in a polymer shell and only a set amount is released little by little. In this connection, a sustained-release action is involved, in which further deliveries of the constituent are made from the capsules, which has decomposed, so that a constant level of photoinitiator is achieved in the continuous phase.

### DESCRIPTION OF THE INVENTION

Preference is given, according to the invention, to acylphosphine oxides of the general formula (I)

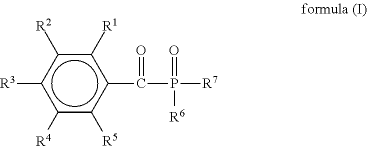

in which

R¹, R², R³, R⁴ and R⁵ can be, independently of one another, hydrogen, halogen, C₁-C₂₀-alkyl, cyclopentyl, cyclohexyl, C₂-C₁₂-alkenyl, C₂-C₁₈-alkyl interrupted by one or more oxygen atoms, C₁-C₄-alkyl substituted by phenyl, unsubstituted phenyl or phenyl substituted with one or two C₁-C₄-alkyl and/or C₁-C₄-alkoxy, and

R⁶ and R⁷ are

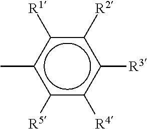

and can exhibit different substituents for R⁶ and R⁷, independently of one another,

it being possible for R¹′, R^{2′}, R^{3′}, R^{4′} and R^{5′} to be, independently of one another, hydrogen, halogen, C₁-C₂₀-alkyl, cyclopentyl, cyclohexyl, C₂-C₁₂-alkenyl, C₂-C₁₈-alkyl interrupted by one or more oxygen atoms, C₁-C₄-alkyl substituted by phenyl, unsubstituted phenyl or phenyl substituted with one or two C₁-C₄-alkyl and/or C₁-C₄-alkoxy.

Preference is furthermore given to bisacylphosphine oxides of the general formula (II),

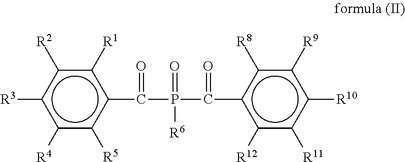

be used in which

R¹, R², R³, R⁴, R⁵, R⁸, R⁹, R¹⁰, R¹¹ and R¹² can be, independently of one another, hydrogen, halogen, C₁-C₂₀-alkyl, cyclopentyl, cyclohexyl, C₂-C₁₂-alkenyl, C₂-C₁₈-alkyl interrupted by one or more oxygen atoms, C₁-C₄-alkyl substituted by phenyl, unsubstituted phenyl or phenyl substituted with one or two C₁-C₄-alkyl and/or C₁-C₄-alkoxy, and

R⁶ can be

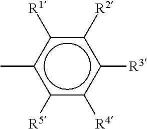

it being possible for R^{1′}, R^{2′}, R^{3′}, R^{4′} and R^{5′} to be, independently of one another, hydrogen, halogen, C₁-C₂₀-alkyl, cyclopentyl, cyclohexyl, C₂-C₁₂-alkenyl, C₂-C₁₈-alkyl interrupted by one or more oxygen atoms, C₁-C₄-alkyl substituted by phenyl, unsubstituted phenyl or phenyl substituted with one or two C₁-C₄-alkyl and/or C₁-C₄-alkoxy.

Acylphosphine oxides and bisacylphosphine oxides are commercially available. Thus, the acylphosphine oxide (2,4,6-trimethylbenzoyl)diphenylphosphine oxide is sold commercially, for example under the trade names Darocur® TPO and Lucirin® TPO, and the bisacylphosphine oxide bis(2,4,6-trimethylbenzoyl)phenylphosphine oxide is commercially available, for example under the trade name Irgacure® 819.

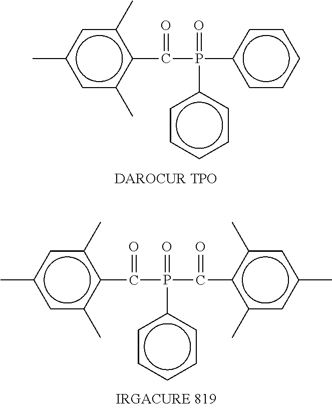

Darocur® and Irgacure® are registered trade marks of Ciba Specialities and Lucirin® is a registered trade mark of BASF AG.

In addition, acylphosphine oxides and bisacylphosphine oxides are relatively simply accessible synthetically. Thus, the synthesis of acylphosphine oxides is described in detail in, e.g., DE 2909992. In this connection, an acid halide is reacted with an appropriate phosphine. In the case of (2,4,6-trimethylbenzoyl)diphenylphosphine oxide, 2,4,6-trimethylbenzoyl chloride is reacted with methoxydiphenylphosphine. The synthesis of bisacylphosphine oxides is described in detail in DE 19708294, for example. Thus, compounds of the formula (II) can be prepared by double acylation of a primary phosphine with at least 2 equivalents of an acid chloride in the presence of a base and subsequent oxidation of the diacylphosphine obtained to give the corresponding bisacylphosphine oxide. Thus, in the case of bis(2,4,6-trimethylbenzoyl)phenylphosphine oxide, dichlorophenylphosphine is first reacted with elemental lithium. Bis(2,4,6-trimethylbenzoyl)phenylphosphine is then formed, from the dilithium phenylphosphide produced, by addition of 2 equivalents of 2,4,6-trimethylbenzoyl chloride and is subsequently oxidized by means of H₂O₂ to give bis(2,4,6-trimethylbenzoyl)phenylphosphine oxide.

Preference is additionally given to amine coinitiators of the general formula (III),

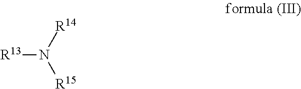

in which

R¹³ can be C₁-C₂₀-alkyl, C₂-C₁₈-alkyl interrupted by one or more oxygen atoms or C₁-C₄-alkyl substituted by CN or OH,

R¹⁴ and R¹⁵ can be, independently of one another, C₁-C₂₀-alkyl, C₂-C₁₈-alkyl interrupted by one or more oxygen atoms, C₁-C₄-alkyl substituted by CN or OH, unsubstituted phenyl or phenyl substituted with one, two or three C₁-C₆-alkyl and/or with alkoxycarbonyl.

The term “amine coinitiators” is preferably understood to mean trifunctional amines which promote rapid curing under the action of light, provided that they are used in combination with a photosensitizer. Examples of such a photosensitizer are benzophenones.

Preference is additionally given to amine coinitiators of the general formula (III),

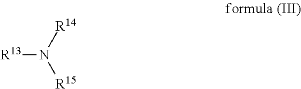

in which

R¹³ can be C₁-C₂₀-alkyl, C₂-C₁₈-alkyl interrupted by one or more oxygen atoms or C₁-C₄-alkyl substituted by CN or OH,

R¹⁴ and R¹⁵ can be, independently of one another, C₁-C₂₀-alkyl, C₂-C₁₈-alkyl interrupted by one or more oxygen atoms, C₁-C₄-alkyl substituted by CN or OH, unsubstituted phenyl or phenyl substituted with one, two or three C₁-C₆-alkyl and/or with alkoxycarbonyl.

Particularly preferred amine coinitiators of the general formula (III) are esters of p-dimethylaminobenzoic acid, such as, for example, ethyl 4-dimethylaminobenzoate or 2-ethylhexyl 4-dimethylaminobenzoate, or benzophenone derivatives, such as 4-(dimethylamino)benzophenone, or aniline derivatives, such as 3,5,N,N-tetramethylaniline, 4,N,N-trimethylaniline, 4-(tert-butyl)-N,N-dimethylaniline, N-cyanoethyl-N-methylaniline or 2,4,6,N,N-pentamethylaniline, but also nonaromatic amines, such as triethanolamine or N-methyldiethanolamine.

Tertiary amine coinitiators are commercially available; 2-ethylhexyl 4-dimethylaminobenzoate is sold, e.g., under the name Genocure® EHA or ethyl 4-dimethylaminobenzoate is sold under the name Genocure® EPD (Rahn Chemie, Zurich).

The term “phenyl substituted with alkoxycarbonyl” is understood to mean compounds of the formula (IV)

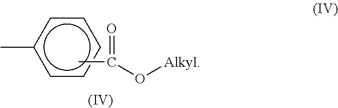

This can act as an R¹⁴ and/or R¹⁵ substituent for the amine coinitiators of the formula (III). The representation means that the phenyl radical is bonded to the nitrogen of the tertiary amine and the alkoxycarbonyl group can be bonded to the phenyl ring at any position. The number of the carbon atoms of the alkyl group lies in the range from C₁ to C₈.

(Meth)acrylic compounds are preferably possible as polymers for the shell of the photoinitiator system. Particular preference is given according to the invention to polymethacrylates or polymethacrylamides or blends thereof, it being possible to adjust the degree of crosslinking of the polymer and accordingly the rate of release of photoinitiator by addition of specific amounts of polyfunctionalized (meth)acrylic compounds.

The microcapsules according to the invention are prepared via the miniemulsion route (N. Bechthold, F. Tiarks, M. Willert, K. Landfester and M. Antonietti, “Miniemulsion polymerization: Applications and new materials”, Macromol. Symp., 2000, 151, 549-555), particularly preferably via the direct miniemulsion (oil-in-water). For this, a two-phase system, consisting of a mixture of
i) the photoinitiator according to formula (I), (II) or (III) to be encapsulated, or a mixture thereof,
ii) at least one radically polymerizable monomer,
iii) at least one ultrahydrophobic compound and
iv) at least one initiator for the thermal initiation of the radical polymerization,
v) is added to a mixture of water and at least one surfactant and
vi) subsequently treated with ultrasound in order to form a stable miniemulsion.

The monomer droplets formed are polymerized by subsequent heating, resulting in the formation of the microcapsules according to the invention. After purification and drying, the microcapsules obtained in this way can be redispersed in the dental material according to the invention.

The miniemulsions according to the invention preferably comprise, based on the total weight of the miniemulsion:
from 0.1 to 40% by weight of the photoinitiator according to formula (I), (II) or (III) to be encapsulated, or a mixture thereof, particularly preferably from 0.5 to 20% by weight,
from 1.0 to 80% by weight of polymerizable monomer, particularly preferably from 2.0 to 50% by weight, between 0.1 and 3% by weight, particularly preferably between 0.5 and 2% by weight, of the ultrahydrophobic compound,
from 0.05 to 5% by weight of at least one initiator for the thermal initiation of the radical polymerization, particularly preferably from 0.1 to 3% by weight,
from 0.01 to 5% by weight of at least one surfactant, particularly preferably from 0.1 to 3% by weight,

the figures for the % by weight adding up each time to 100% by weight.

Use may be made, as radically polymerizable monomers, of mono-or polyfunctional (meth)acrylates or (meth)acrylamides ((meth)acrylic compounds). The term “monofunctional (meth)acrylic compounds” is understood to mean compounds with one (meth)acrylic group and the term “polyfunctional (meth)acrylic compounds” is understood to mean compounds with two or more, preferably 2 or 3, (meth)acrylic groups. Polyfunctional monomers have crosslinking properties.

Preferred monofunctional (meth)acrylic compounds are commercially available monofunctional monomers, such as methyl, ethyl, butyl, benzyl, furfuryl or phenyl (meth)acrylate, and also 2-hydroxyethyl (meth)acrylate or 2-hydroxypropyl (meth)acrylate.

Preferred polyfunctional (meth)acrylic compounds are bisphenol A di(meth)acrylate, Bis-GMA (an addition product of methacrylic acid and bisphenol A diglycidyl ether), ethoxylated bisphenol A di(meth)acrylate, UDMA (an addition product of 2-hydroxyethyl methacrylate and 2,2,4-trimethylhexamethylene diisocyanate), di-, tri- or tetraethylene glycol di(meth)acrylate, trimethylolpropane tri(meth)acrylate, pentaerythrityl tetra(meth)acrylate, and also butanediol di(meth)acrylate, 1,10-decanediol di(meth)acrylate or 1,12-dodecanediol di(meth)acrylate, crosslinking pyrrolidones, such as, e.g., 1,6-bis(3-vinyl-2-pyrrolidonyl)hexane, or commercially available bisacrylamides, such as methylene- or ethylenebisacrylamide, bis(meth)acrylamides, such as, e.g., N,N′-diethyl-1,3-bis-(acrylamido)propane, 1,3-bis(methacrylamido)propane, 1,4-bis(acrylamido)butane or 1,4-bis(acryloyl)piperazine, which can be synthesized by reaction of the corresponding diamines with (meth)acryloyl chloride.

Use is made, as ultrahydrophobic compounds, of very nonpolar organic compounds, such as, in particular, aliphatic or aromatic hydrocarbons. Preference is given, among aliphatic hydrocarbons, in particular to C₆-C₂₀-alkanes. Those chosen from the group composed of aliphatic hydrocarbons, in particular consisting of hexadecane, are very particularly preferred.

Ionic and nonionic amphiphilic compounds well known for the preparation of emulsions are used as surfactant. Use is preferably made of the anionic sodium dodecyl sulfate, the cationic cetyltrimethylammonium chloride, the nonionic C₁₆-EO₅₀ (Lutensol® AT50) or block copolymers.

In order to guarantee that the polymerization of the relatively hydrophilic monomer (e.g., methyl methacrylate) takes place only in the droplets comprising photoinitiator and that no secondary particles are formed, a hydrophobic diazo compound is preferably used as thermal initiator. Through the thermal initiation, the monomer is polymerized in such a way that a shell is formed at the interface of the droplet with the continuous phase. The monomer and the photoinitiator according to formula (I) or (II) are miscible in the miniemulsion stage but phase separation occurs during the polymerization. Due to the hydrophobic nature of the photoinitiator according to formula (I) or (II) and the more hydrophilic nature of the polymer, microcapsules with a core/shell geometry are formed, the photoinitiator being situated in the core, encapsulated in a polymer shell.

Preferred diazo initiators are the commercially available compounds for the initiation of radical polymerizations, such as, e.g., AIBN or 2,2′-azobis(2-methylbutyronitrile).

The photoinitiator systems described are suitable above all for the use for self-etching, self-conditioning dental materials. They are particularly suitable for the use of dentin/enamel adhesives. Such dental materials are correspondingly suitable preferably as self-adhering coating materials and/or self-conditioning fixing cements, particularly preferably as self-etching adhesives.

Dental materials can preferably be prepared, preferably in the form of self-etching, self-conditioning dental materials, in the following composition with the microcapsules according to the invention:
a) from 0.05 to 20.0% by weight, preferably from 0.5 to 15% by weight and particularly preferably from 0.5 to 10% by weight of microencapsulated photoinitiator of the formula (I) or (II) or (III) or mixtures thereof;
b) from 0 to 10.0% by weight, preferably from 0.05 to 7.5% by weight and particularly preferably from 0.1 to 5.0% by weight of -diketone;
c) from 0 to 10.0% by weight, preferably from 0.1 to 5% by weight, of non-microencapsulated photoinitiator of the formula (I) or (II) or (III) or mixtures thereof;
d) from 5 to 95% by weight, preferably from 5 to 85% by weight and particularly preferably from 5 to 70% by weight of mono- or polyfunctional monomer;
e) from 0 to 60% by weight, preferably from 5 to 50% by weight and particularly preferably from 5 to 45% by weight of acidic radically polymerizable monomer;
f) from 0 to 80% by weight, preferably from 5 to 60% by weight and particularly preferably from 10 to 40% by weight of solvent;
g) from 0 to 85% by weight of filler, particularly preferably from 1 to 75% by weight, and
h) from 0.01 to 5.0% by weight of pigments, inhibitors and stabilizers,

the figures for the % by weight adding up each time to 100% by weight.

Dental materials, preferably in the form of self-etching, self-conditioning dental materials, for use as adhesive or coating material preferably comprise from 1 to 30% by weight of filler and dental materials for use as cement preferably comprise from 20 to 85% by weight of filler. Moreover, in adhesives and coating materials, preferably from 5 to 60% by weight of solvent is used. Preferred solvents are water, methanol, ethanol, isopropanol, ethyl acetate, acetone and mixtures thereof.

The dental materials according to the invention obtained using the photoinitiators described can comprise, as radically polymerizable monomers, mono- or polyfunctional (meth)acrylates or (meth)acrylamides ((meth)acrylic compounds). The term “monofunctional (meth)acrylic compounds” is understood to mean compounds with one (meth)acrylic group and the term “polyfunctional (meth)acrylic compounds” is understood to mean compounds with two or more, preferably 2 or 3, (meth)acrylic groups. Polyfunctional monomers have crosslinking properties.

Preferred monofunctional (meth)acrylic compounds are commercially available monofunctional monomers, such as methyl, ethyl, butyl, benzyl, furfuryl or phenyl (meth)acrylate, and also 2-hydroxyethyl (meth)acrylate or 2-hydroxypropyl (meth)acrylate.

Particular preference is given to hydrolysis-stable monomers, such as hydrolysis-stable mono(meth)acrylates, e.g. mesityl methacrylate, or 2-(alkoxymethyl)acrylic acids, e.g. 2-(ethoxymethyl)acrylic acid, 2-(hydroxymethyl)acrylic acid, N-monosubstituted or N,N-disubstituted acrylamides, such as, e.g., N-ethylacrylamide, N,N-dimethylacrylamide, N-(2-hydroxyethyl)acrylamide or N-methyl-N-(2-hydroxyethyl)-acrylamide, and N-monosubstituted methacrylamides, such as, e.g., N-ethylmethacrylamide or N-(2-hydroxyethyl)-methacrylamide, and also, in addition, N-vinylpyrrolidone and allyl ether. These monomers are liquid at ambient temperature and are accordingly also suitable as diluents.

Preferred polyfunctional monomers are bisphenol A di(meth)acrylate, Bis-GMA (an addition product of methacrylic acid and bisphenol A diglycidyl ether), ethoxylated bisphenol A di(meth)acrylate, UDMA (an addition product of 2-hydroxyethyl methacrylate and 2,2,4-trimethylhexamethylene diisocyanate), di-, tri- or tetraethylene glycol di(meth)acrylate, trimethylolpropane tri(meth)acrylate, pentaerythrityl tetra(meth)acrylate, and also butanediol di(meth)acrylate, 1,10-decanediol di(meth)acrylate or 1,12-dodecanediol di(meth)acrylate.

Particular preference is given to hydrolysis-stable crosslinker monomers, such as, e.g., crosslinking pyrrolidones, such as, e.g., 1,6-bis(3-vinyl-2-pyrrolidonyl)hexane, or commercially available bisacrylamides, such as methylene- or ethylenebisacrylamide, bis(meth)-acrylamides, such as, e.g., N,N′-diethyl-1,3-bis-(acrylamido)propane (DBAP), 1,3-bis(methacrylamido)propane, 1,4-bis(acrylamido)butane or 1,4-bis(acryloyl)piperazine, which can be synthesized by reaction of the corresponding diamines with (meth)acryloyl chloride.

The dental materials according to the invention obtained by the use of the photoinitiators described preferably also comprise at least one radically polymerizable monomer comprising an acid group. Preferred acid groups are carboxylic acid groups, phosphonic acid groups, phosphate groups and/or sulfonic acid groups, these groups being able to be present in the acid form, as anhydride or in the form of an ester. Particular preference is given to monomers with phosphonic acid groups or phosphate groups. The monomers can exhibit one or more acid groups; preference is given to compounds with from 1 to 2 acid groups.

Preferred polymerizable carboxylic acids are maleic acid, acrylic acid, methacrylic acid, 2-(hydroxymethyl)acrylic acid, 4-(meth)acryloyloxyethyltrimellitic acid or the corresponding anhydride, 10-methacryloyloxydecylmalonic acid, N-(2-hydroxy-3-methacryloyloxypropyl)-N-phenylglycine, N-acryloylaspartic acid (AAA) and 4-vinylbenzoic acid.

Preferred phosphonic acid monomers are vinylphosphonic acid, 4-vinylphenylphosphonic acid, 4-vinylbenzylphosphonic acid, 2-methyacryloyloxyethylphosphonic acid, 2-methacrylamidoethylphosphonic acid, 4-methacrylamido-4-methylpentylphosphonic acid, 2-[4-(dihydroxyphosphoryl)-2-oxabutyl]acrylic acid, 2,4,6-trimethylphenyl 2-[2-(dihydroxyphosphoryl)ethoxymethyl]acrylate and ethyl 2-[2-(dihydroxyphosphoryl)ethoxymethyl]acrylate (EDPEA).

Preferred acidic polymerizable phosphoric acid esters are 2-methacryloyloxypropyl mono- and dihydrogenphosphate, 2-methacryloyloxyethyl mono- and dihydrogenphosphate, 2-methacryloyloxyethyl phenyl hydrogenphosphate, dipentaerythritol pentamethacryloyloxy phosphate, 10-methacryloyloxydecyl dihydrogenphosphate, dipentaerythritol pentamethacryloyloxy phosphate, phosphoric acid mono(1-acryloylpiperidin-4-yl) ester, 6-(methacrylamido)hexyl dihydrogenphosphate, 1,3-bis(N-acryloyl-N-propylamino)prop-2-yl dihydrogenphosphate and 1,3-bis(methacrylamido)prop-2-yl dihydrogenphosphate (BMPP).

Preferred polymerizable sulfonic acids are vinylsulfonic acid, 4-vinylphenylsulfonic acid or 3-(methacrylamido)propylsulfonic acid.

Use may also be made, as mono- or polyfunctional monomer, of polymerizable compounds exhibiting an antimicrobial action, such as, e.g., 12-methacryloyloxydodecylpyridinium bromide; particular preference is given to “macromers”, which comprise a polymeric spacer between the polymerizable group and the group having an antimicrobial action.

Furthermore, the dental materials can comprise organic or inorganic particulate fillers to improve the mechanical properties or to adjust the viscosity. Preferred inorganic particulate fillers are amorphous spherical materials based on oxides, such as ZrO₂ and TiO₂, nanoparticulate or microfine fillers, such as fumed silica, nanoparticulate Al₂O₃, Ta₂O₅, Yb₂O₃, ZrO₂, Ag or TiO₂, or mixed oxides of SiO₂, ZrO₂ and/or TiO₂, or precipitated silica, and also minifillers, such as quartz powder, glass ceramic powder or glass powder with a mean particle size of 0.01 to 5 m, and also X-ray opaque fillers, such as ytterbium trifluoride or nanoparticulate barium sulfate. Particularly suitable are fillers surface-modified with polymerizable groups.

In addition, the dental materials prepared with the photoinitiators described can comprise one or more additional additives chosen from stabilizers, flavoring agents, dyes, pigments, additives which release fluoride ions, optical brighteners, plasticizers and/or UV absorbers. A preferred UV absorber is 2-hydroxy-4-methoxybenzophenone; preferred stabilizers are 2,6-di(tert-butyl)-4-cresol and 4-methoxyphenol.

Another subject matter of the invention is the starting mixtures for the preparation of microcapsules for the dental materials described which comprise
i) photoinitiator(s) to be encapsulated,
ii) at least one polymerizable monomer,
iii) at least one ultrahydrophobic compound and
iv) at least one initiator for the thermal initiation of the radical polymerization.

Reference may be made, with regard to the details concerning the amounts and materials of the components i)-iv), to the above description.

The invention is more fully explained below with the help of examples.

### EXAMPLES

Examples of the preparation of the microencapsulated photoinitiators (I) and (II)

### Example 1

### PMMA Encapsulation of Photoinitiator (I)

To prepare the disperse phase, 1.0 g of (2,4,6-trimethylbenzoyl)diphenylphosphine oxide (Lucirin® TPO), 5.0 g of methyl methacrylate, 250 mg of hexadecane and 100 mg of 2,2′-azobis(2-methylbutyl-1-nitrile) are mixed in a 50 ml glass beaker and stirred with the exclusion of light until the Lucirin® TPO has completely dissolved. For the continuous phase, 72 mg of sodium dodecyl sulfate are dissolved in 24.0 g of demineralized water. Subsequently, the continuous phase is added, with the exclusion of light and continuous stirring, to the disperse phase and stirred at 2000 rev.min⁻¹ for 1 h. After the removal of the magnetic stirrer bar, the macroemulsion formed is miniemulsified by means of an ultrasonic probe (½″ tip) at 90% amplitude in an ice bath for a sonification time of 2 min. The sample is transferred into a 50 ml amber-colored flask, the flask is firmly closed and polymerization is carried out overnight with continuous stirring at 1000 rev.min⁻¹ in an oil bath already preheated to 72° C. After removing from the oil bath, the suspension is filtered while warm and freeze dried. To purify and to remove nonencapsulated Lucirin® TPO, the capsules are placed in a Büchner funnel, 30 ml of ethanol are added and vacuum is subsequently applied. The remaining ethanol is removed by renewed freeze drying, thereby preventing the capsules from swelling. To determine the Lucirin® TPO content, 60 mg of the dried capsule material are dissolved with 2.0 mg of pyrazine (1,4-diazine) in 1 ml of CDCl₃ and measured in an amber-colored NMR glass tube. In the ¹H NMR spectrum, pyrazine acts as reference for the quantitative determination of the Lucirin® TPO present. The particle size, determined by dynamic light scattering (DLS), is 132 nm; the proportion of Lucirin in the capsules is 28.71% by weight of the Lucirin originally used (corresponds to a pure Lucirin proportion of the capsules of approximately 5% by weight).

### Example 2

### Copolymer Encapsulation of Photoinitiator (I)

Instead of methyl methacrylate (MMA) as sole monomer, a mixture of butyl acrylate (BA) and methyl methacrylate is used as monomer mixture. The amount is 5 g. The particle size, determined by DLS, the lucirin content and the glass transition temperature (T_{g}), determined by calorimetry, of the samples are summarized in table 1. It can clearly be seen, in table 1, that the glass transition temperature of the polymer shell of the particles is reduced through the addition of butyl acrylate.

TABLE 1
Copolymer-encapsulated Lucirin samples

| | | | | Lucirin |
|---|---|---|---|---|
| | Monomer | Particle | | content* [% |
| Sample | mixture [g] | size [nm] | T_{g} [° C.] | by weight] |
| 2A | 2.4 g MMA | 112 | −1.1 | 83.78 (13) |
| | 2.6 g BA | | | |
| 2B | 2.6 g MMA | 132 | 6.0 | 97.02 (15) |
| | 2.4 g BA | | | |
| 2C | 3.0 g MMA | 108 | 28.9 | 77.79 (12.5) |
| | 2.0 g BA | | | |
| 2D | 3.4 g MMA | 106 | 32.0 | 70.60 (11.5) |
| | 1.6 g BA | | | |

*first value: based on amount of Lucirin weighed out; second value in brackets: based on final particle

### Example 3

### PMMA Encapsulation of Photoinitiator (I) with Molecular Weight Regulation

To reduce the molecular weight, use is made of a mixture of methyl methacrylate and butyl mercaptan (BuSH). The particle size, the Lucirin content and the molecular weight (M_{w}), determined by GPC, of the samples are given in table 2.

TABLE 2
Encapsulated Lucirin in PMMA shell with
molecular weight regulator

| | | | | Lucirin |
|---|---|---|---|---|
| | Monomer | Particle | M_{w} | content* [% |
| Sample | mixture [g] | size [nm] | [g/mol] | by weight] |
| 3A | 5.0 g MMA | 109 | 1.5 · 10⁵ | 55.89 (9) |
| | 6.2 mg BuSH | | | |
| 3B | 5.0 g MMA | 126 | 4.13 · 10⁴ | 42.00 (7) |
| | 26.8 mg BuSH | | | |
| 3C | 5.0 g MMA | 134 | 2.95 · 10⁴ | 68.75 (11) |
| | 48.7 mg BuSH | | | |

*first value: based on amount of Lucirin weighed out; second value in brackets: based on final particle

### Example 4

### Crosslinked PMMA Encapsulation of Photoinitiator (I)

A mixture of methyl methacrylate and crosslinking agent DBAP was used as monomer mixture. The amount of the crosslinking agent can be varied within a wide range. The characteristics are given in table 3.

TABLE 3
Lucirin encapsulation in crosslinked PMMA shell

| Sample | Monomer | Particle | Lucirin |
|---|---|---|---|
| description | mixture [g] | size [nm] | content* [%] |
| 4A | 4.5 g MMA | 107 | 89.81 (14) |
| | 0.5 g DBAP | | |
| 4B | 4.0 g MMA | 189 | 66.35 (11) |
| | 1.0 g DBAP | | |

*first value: based on amount of Lucirin weighed out; second value in brackets: based on final particle

### Example 5

### PMMA Encapsulation of Photoinitiator (II)

Encapsulation of bis(2,4,6-trimethylbenzoyl)phenylphosphine oxide (Irgacure® 819)

For the preparation of the disperse phase, 0.7 g of Irgacure® 819, 5.3 g of methyl methacrylate, 250 mg of hexadecane and 100 mg of 2,2′-azobis(2-methylbutyronitrile) are mixed in a 50 ml glass beaker (tall form) and are stirred with the exclusion of light until the Irgacure® 819 has completely dissolved. For the continuous phase, 72 mg of sodium dodecyl sulfate (SDS) are dissolved in 24.0 g of demineralized water. Subsequently, the continuous phase is added, with the exclusion of light and continuous stirring, to the disperse phase and stirred at 2000 rev.min⁻¹ for 1 h (likewise with the exclusion of light). After the removal of the magnetic stirrer bar, the macroemulsion formed is miniemulsified by means of an ultrasonic probe (½″ tip) at 90% amplitude in an ice bath for a sonification time of 2 min. The sample is transferred into a 50 ml amber-colored flask, the flask is firmly closed and polymerization is carried out overnight with continuous stirring at 1000 rev.min⁻¹ in an oil bath already preheated to 72° C. After removing from the oil bath, the suspension is filtered while warm and freeze dried. The particle size of the particles, determined by dynamic light scattering, is approximately 170 nm.

Kinetics of release of the encapsulated Lucirin® TPO:

100.0 mg of freeze-dried purified capsules prepared according to example 1 or 4A are dispersed in 1.0 ml of isopropanolic pyrazine (1,4-diazine) solution for a defined time and the capsules are subsequently filtered off through a syringe filter with a prefilter. The filtrate is measured with an external standard of D₂O (deuterated water, deuterium oxide) in a ¹H NMR spectrometer. Pyrazine acts in this connection as reference for the quantitative determination of the amount of Lucirin® TPO released. In this connection, the maximum content of Lucirin® TPO present in the capsules is rated as 100% release.

The release of the initiator from the capsules prepared according to example 1 and example 4A was investigated in 2-propanol (FIG. 1). It can be seen that the release time increases from 10 min for pure PMMA particles to a release time of 90 min for the crosslinked PMMA particles. This shows that the kinetics of release of the encapsulated initiator can be influenced simply and within wide limits by varying the polymer shell. It is thus possible for the rate of release of the encapsulated initiator systems to conform to the monomer mixtures used each time.

### Example of the Preparation of the Microencapsulated Coinitiator (III)

### Example 6

### PMMA Encapsulation of Amine Coinitiator (III)

To prepare the disperse phase, 1.0 g of ethyl p-dimethylaminobenzoate (EMBO), 4.5 g of methyl methacrylate, 0.5 g of N,N′-diethyl-N,N′-diacryloylpropylenediamine, 250 mg of hexadecane and 100 mg of 2,2′-azobis(2-methylbutyl-1-nitrile) are mixed in a 50 ml glass beaker (tall form) and stirred until the EMBO has completely dissolved. For the continuous phase, 72 mg of sodium dodecyl sulfate are dissolved in 24.0 g of demineralized water. Subsequently, the continuous phase is added, with continuous stirring, to the disperse phase and stirred at 2000 rev.min⁻¹ for 1 h. After the removal of the magnetic stirrer bar, the macroemulsion formed is miniemulsified by means of an ultrasonic probe (½″ tip) at 90% amplitude in an ice bath for a sonification time of 2 min. The sample is transferred into a 50 ml flask, the flask is firmly closed and polymerization is carried out overnight with continuous stirring at 1000 rev.min⁻¹ in an oil bath already preheated to 72° C. After removing from the oil bath, the suspension is filtered while warm and freeze dried.

The diameter of the capsules, determined by DLS, is 114 nm; the content of Lucirin in the capsules was determined as being 15.7%.

### Examples of Dental Materials

The encapsulated initiator systems according to the invention were incorporated in the following monomer mixtures subsequently used for adhesives in the dental field:

Dentin Adhesive Mixture 1:

To prepare the monomer mixture, 15% of 2-hydroxyethyl methacrylate, 10% of EDPEA, 30% of Bis-GMA, 20% of UDMA and 25% of ethanol are mixed with a magnetic stirrer and stirred for 30 min.

Dentin Adhesive Mixture 2:

To prepare the monomer mixture, 25% of water, 50% of DBAP, 15% of BMPP and 10% of AAA are mixed with a magnetic stirrer and stirred for 30 min.

### Example 7

Dentin Adhesive 1, Comprising Encapsulated Photoinitiator (I)

To prepare the adhesive formulation (7a), 99% of dentin adhesive mixture 1 and 1.0% of encapsulated Lucirin® TPO according to example 4A are mixed with a magnetic stirrer. The solution obtained is stored at 42° C. in a drying cupboard and, after regular time intervals, the polymerization time is determined according to ISO 6874 (1988). The operation of the initiator in the adhesive formulation can be fully monitored using the polymerization time. If the polymerization slows down and accordingly the polymerization time lengthens, this is a clear indication of decomposition of the initiator system in the formulation. The polymerization times are listed in table 4.

An adhesive formulation (7B) consisting of 99.85% of dentin adhesive mixture 2 and 0.15% of Lucirin® TPO acts as comparison.

As emerges from table 4, the encapsulated initiator used in formulation 7A shows a markedly improved storage stability in comparison with the nonencapsulated system. Due to the slow release of the initiator in formulation 7A, only a relatively slow polymerization and accordingly a long polymerization time for the formulation can be observed at the beginning of the investigation into storage stability. This effect can be circumvented by the addition of a defined amount of nonencapsulated initiator, as is shown in example 7C. To prepare the adhesive formulation (7C), 99.57% of dentin adhesive mixture 1, 0.33% of encapsulated Lucirin® TPO according to example 4A and 0.10% of nonencapsulated Lucirin® TPO are mixed with a magnetic stirrer. As is shown in table 4, the polymerization time of formulation 7C remains constant during the entire span of the investigation.

TABLE 4

| | Storage life (42° C.) | | | | | |
|---|---|---|---|---|---|---|
| Polymerization | 0 | 14 | 28 | 56 | 84 | 112 |
| time (s) | Days | Days | Days | Days | Days | Days |
| Formulation 7A | >60 s | 30 s | 15 s | 13 s | 12 s | 13 s |
| Formulation 7B | 12 s | 13 s | 12 s | 19 s | 34 s | 50 s |
| Formulation 7C | 15 s | 13 s | 12 s | 11 s | 13 s | 12 s |

### Example 8

Dentin adhesive 2, comprising encapsulated photoinitiator (I)

To prepare the adhesive formulation (8A), 98.67% of dentin adhesive mixture 2 and 1.33% of encapsulated Lucirin® TPO according to example 4A are mixed with a magnetic stirrer. The solution obtained is stored at 42° C. in a drying cupboard and, after regular time intervals, the polymerization time is determined. The polymerization times are listed in table 5.

An adhesive formulation (8B) consisting of 99.8% of dentin adhesive mixture 2 and 0.2% of Lucirin® TPO acts as comparison.

As emerges from table 5, the encapsulated initiator used in formulation 8A shows a markedly improved storage stability in comparison with the nonencapsulated system. Due to the slow release of the initiator in formulation 8A, only a relatively slow polymerization and accordingly a long polymerization time for the formulation can be observed at the beginning of the investigation into storage stability. This effect can be circumvented by the addition of a defined amount of nonencapsulated initiator, as is shown in example 8C. To prepare the adhesive formulation (8C), 99.23% of dentin adhesive mixture 2, 0.67% of encapsulated Lucirin® TPO according to example 4A and 0.10% of nonencapsulated Lucirin® TPO are mixed with a magnetic stirrer. As is shown in table 5, the polymerization time of formulation 8C remains constant during the entire span of the investigation.

TABLE 5

| | Storage life (42° C.) | | | | | |
|---|---|---|---|---|---|---|
| Polymerization | 0 | 14 | 28 | 56 | 84 | 112 |
| time (s) | Days | Days | Days | Days | Days | Days |
| Formulation 8A | >60 s | 57 s | 35 s | 24 s | 26 s | 25 s |
| Formulation 8B | 27 s | 25 s | 26 s | 30 s | 45 s | 58 s |
| Formulation 8C | 28 s | 26 s | 25 s | 27 s | 23 s | 25 s |

### Example 9

Dentin Adhesive 1, Comprising Encapsulated Amine Coinitiator (III)

To prepare the adhesive formulation (9A), 96.47% of dentin adhesive mixture 1, 0.2% of camphorquinone and 3.33% of encapsulated ethyl p-dimethylaminobenzoate (EMBO) according to example 6 are mixed with a magnetic stirrer. The solution obtained is stored at 42° C. in a drying cupboard and, after regular time intervals, the polymerization time is determined. The polymerization times are listed in table 6.

An adhesive formulation (9B) consisting of 99.3% of dentin adhesive mixture 1, 0.2% of camphorquinone and 0.5% of ethyl p-dimethylaminobenzoate (EMBO) acts as comparison.

As emerges from table 6, the encapsulated initiator used in formulation 9A shows a markedly improved storage stability in comparison with the nonencapsulated system. Due to the slow release of the initiator in formulation 9A, only a relatively slow polymerization and accordingly a long polymerization time for the formulation can be observed at the beginning of the investigation into storage stability. This effect can be circumvented by the addition of a defined amount of nonencapsulated initiator, as is shown in example 9C. To prepare the adhesive formulation (9C), 97.88% of dentin adhesive mixture 1, 0.2% of camphorquinone, 1.67% of encapsulated ethyl p-dimethylaminobenzoate (EMBO) according to example 6 and 0.25% of nonencapsulated EMBO are mixed with a magnetic stirrer. As is shown in table 6, the polymerization time of formulation 9C remains constant during the entire span of the investigation.

TABLE 6

| | Storage life (42° C.) | | | | | |
|---|---|---|---|---|---|---|
| Polymerization | 0 | 14 | 28 | 56 | 84 | 112 |
| time (s) | Days | Days | Days | Days | Days | Days |
| Formulation 9A | 50 s | 31 s | 25 s | 27 s | 26 s | 27 s |
| Formulation 9B | 26 s | 25 s | 27 s | 35 s | 43 s | 50 s |
| Formulation 9C | 26 s | 27 s | 24 s | 25 s | 27 s | 25 s |

### Example 10

Dentin Adhesive 2, Comprising Encapsulated Amine Coinitiator (III)

To prepare the adhesive formulation (10A), 96.47% of dentin adhesive mixture 2, 0.2% of camphorquinone and 3.33% of encapsulated ethyl p-dimethylaminobenzoate (EMBO) according to example 6 are mixed with a magnetic stirrer. The solution obtained is stored at 42° C. in a drying cupboard and, after regular time intervals, the polymerization time is determined. The polymerization times are listed in table 7.

An adhesive formulation (10B) consisting of 99.3% of dentin adhesive mixture 1, 0.2% of camphorquinone and 0.5% of ethyl p-dimethylaminobenzoate (EMBO) acts as comparison.

As emerges from table 7, the encapsulated initiator used in formulation 10A shows a markedly improved storage stability in comparison with the nonencapsulated system. Due to the slow release of the initiator in formulation 10A, only a relatively slow polymerization and accordingly a long polymerization time for the formulation can be observed at the beginning of the investigation into storage stability. This effect can be circumvented by the addition of a defined amount of nonencapsulated initiator, as is shown in example 10C. To prepare the adhesive formulation (10C), 97.88% of dentin adhesive mixture 2, 0.2% of camphorquinone, 1.67% of encapsulated ethyl p-dimethylaminobenzoate (EMBO) according to example 6 and 0.25% of nonencapsulated EMBO are mixed with a magnetic stirrer. As is shown in table 7, the polymerization time of formulation 10C remains constant during the entire span of the investigation.

TABLE 7

| | Storage life (42° C.) | | | | | |
|---|---|---|---|---|---|---|
| Polymerization | 0 | 14 | 28 | 56 | 84 | 112 |
| time (s) | Days | Days | Days | Days | Days | Days |
| Formulation 10A | >60 s | 45 s | 42 s | 44 s | 46 s | 43 s |
| Formulation 10B | 42 s | 45 s | 46 s | 57 s | >60 s | >60 s |
| Formulation 10C | 47 s | 44 s | 43 s | 46 s | 44 s | 45 s |

1. Microcapsules composed of a shell of polymers and of a core comprising photoinitiators which exhibit acylphosphine oxides, their derivatives or amine coinitiators or mixtures of these compounds.

2. The microcapsules as claimed in claim 1, wherein the photoinitiators comprise acylphosphine oxides of the general formula (I) 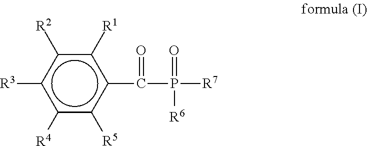
in which
R¹, R², R³, R⁴ and R⁵ can be, independently of one another, hydrogen, halogen, C₁-C₂₀-alkyl, cyclopentyl, cyclohexyl, C₂-C₁₂-alkenyl, C₂-C₁₈-alkyl interrupted by one or more oxygen atoms, C₁-C₄-alkyl substituted by phenyl, unsubstituted phenyl or phenyl substituted with one or two C₁-C₄-alkyl and/or C₁-C₄-alkoxy,
and R⁶ and R⁷ can be 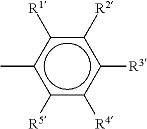
and R⁶ and R⁷ can exhibit, independently of one another, different substituents,
it being possible for R^{1′}, R^{2′}, R^{3′}, R^{4′} and R^{5′} to be, independently of one another, hydrogen, halogen, C₁-C₂₀-alkyl, cyclopentyl, cyclohexyl, C₂-C₁₂-alkenyl, C₂-C₁₈-alkyl interrupted by one or more oxygen atoms, C₁-C₄-alkyl substituted by phenyl, unsubstituted phenyl or phenyl substituted with one or two C₁-C₄-alkyl and/or C₁-C₄-alkoxy.

3. The microcapsules as claimed in claim 1, wherein the photoinitiators exhibit (2,4,6-trimethylbenzoyl)diphenylphosphine oxide.

4. The microcapsules as claimed in claim 1, wherein the photoinitiators comprise bisacylphosphine oxides of the general formula (II) 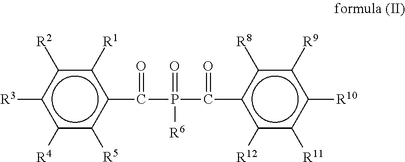
in which
R¹, R², R³, R⁴, R⁵, R⁸, R⁹, R¹⁰, R¹¹ and R¹² can be, independently of one another, hydrogen, halogen, C₁-C₂₀-alkyl, cyclopentyl, cyclohexyl, C₂-C₁₂-alkenyl, C₂-C₁₈-alkyl interrupted by one or more oxygen atoms, C₁-C₄-alkyl substituted by phenyl, unsubstituted phenyl or phenyl substituted with one or two C₁-C₄-alkyl and/or C₁-C₄-alkoxy,
and R⁶ can be 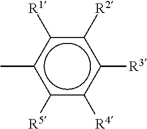
it being possible for R^{1′}, R^{2′}, R^{3′}, R^{4′} and R^{5′} to be, independently of one another, hydrogen, halogen, C₁-C₂₀-alkyl, cyclopentyl, cyclohexyl, C₂-C₁₂-alkenyl, C₂-C₁₈-alkyl interrupted by one or more oxygen atoms, C₁-C₄-alkyl substituted by phenyl, unsubstituted phenyl or phenyl substituted with one or two C₁-C₄-alkyl and/or C₁-C₄-alkoxy.

5. The microcapsules as claimed in claim 1, wherein they comprise bis(2,4,6-trimethylbenzoyl)phenylphosphine oxide.

6. The microcapsules as claimed in claim 1, wherein the photoinitiators comprise amine coinitiators of the general formula (III) 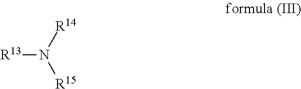
in which
R¹³ can be C₁-C₂₀-alkyl, C₂-C₁₈-alkyl interrupted by one or more oxygen atoms or C₁-C₄-alkyl substituted by CN or OH,
R¹⁴ and R¹⁵ can be, independently of one another, C₁-C₂₀-alkyl, C₂-C₁₈-alkyl interrupted by one or more oxygen atoms, C₁-C₄-alkyl substituted by CN or OH, unsubstituted phenyl or phenyl substituted with one, two or three C₁-C₆-alkyl and/or with alkoxycarbonyl.

7. The microcapsules as claimed in claim 1, wherein the shell comprises, as polymers, methacrylic compounds.

8. The microcapsules as claimed in claim 1, wherein the shell exhibits polymethacrylates or polymethacrylamides.

9. A process for the preparation of the microcapsules as claimed in claim 1, wherein a two-phase system, consisting of a mixture of
i) an encapsulated photoinitiator from one of the abovementioned claims or a mixture thereof,
ii) at least one radically polymerizable monomer,
iii) at least one ultrahydrophobic compound and
iv) at least one initiator for the thermal initiation of the radical polymerization,
v) is added to a mixture of water and at least one surfactant and
vi) subsequently thereto, a stable miniemulsion is formed and is polymerized by subsequent heating.

10. The process as claimed in claim 9, wherein use is made of
from 0.1 to 40% by weight of the photoinitiator to be encapsulated,
from 1.0 to 80% by weight of the polymerizable monomer,
from 0.1 to 2% by weight of the ultrahydrophobic compound,
from 0.05 to 5% by weight of an initiator for the thermal initiation of the radical polymerization and
from 0.01 to 5% by weight of a surfactant,
the figures in % by weight adding up each time to 100% by weight.

11. The process as claimed in claim 10, wherein use is made of
from 0.5 to 20% by weight of the photoinitiator to be encapsulated,
from 2.0 to 50% by weight of the polymerizable monomer,
from 0.5 to 2% by weight of the ultrahydrophobic compound,
from 0.1 to 3% by weight of an initiator for the thermal initiation of the radical polymerization and
from 0.1 to 3% by weight of a surfactant,
the figures in % by weight adding up each time to 100% by weight.

12. The process as claimed in claim 9, wherein use is made, as monomers, of methacrylates or methacrylamides.

13. The process as claimed in claim 9, wherein use is made, as ultrahydrophobic compounds, of aliphatic hydrocarbons or aromatic hydrocarbons.

14. The process as claimed in claim 9, wherein use is made, as ultrahydrophobic compounds, of aliphatic hydrocarbons having C₆-C₂₀-alkanes.

15. The process as claimed in claim 9, wherein use is made, as surfactants, of ionic and nonionic amphiphilic compounds.

16. The use of the microcapsules as claimed in claim 1 for self-etching, self-conditioning dental materials.

17. The use of the microcapsules as claimed in claim 1 for dentin/enamel adhesives.
